(19) United States
(12) Patent Application Publication
(10) Pub. No.: US 2003/0051275 A1
(43) Pub. Date: Mar. 13, 2003

(54) Methods and means for expression of mammalian polypeptides in monocotyledonous plants

(76) Inventors: Paul Christou, Colney (GB); Eva Stoger, Colney (GB); Rainer Fischer, Aachen (DE); Carmen Martin-Vaquero, Colney (GB); Stefan Schillberg, Aachen (DE); Julian K-C Ma, London (GB)

Correspondence Address: FULBRIGHT & JAWORSKI L.L.P., Mary Anne Schofield, Market Square, 801 Pennsylvania Avenue, N.W., Washington, DC, 20004-2615, US

(21) Appl. No.: 10/127,427

(22) Filed: Apr. 23, 2002

Related U.S. Application Data
(63) Continuation of application No. 09/333,527, filed on Jun. 15, 1999, now abandoned.
(60) Provisional application No. 60/089,322, filed on Jun. 15, 1998.

(51) Int. Cl. A01H 5/00; C12N 5/04

(52) U.S. Cl. 800/288; 435/419

(57) ABSTRACT

Rice, wheat and other monocotyledonous plants are transformed with expression cassettes for production of mammalian polypeptides, such as antibodies. Endoplasmic reticulum (ER) retention signals, 5′ untranslated regions and leader peptides are employed in various combinations to provide high expression yield. Multi-chain complexes such as four-chain secretory antibodies are produced by expression of component polypeptides from separate vectors all introduced into the same cell by transformation.

This application claims priority from U.S. provisional application serial No. 60/089,322 filed Jun. 15, 1998.

The present invention relates to expression of transgenes in plants, especially monocots, in particular non-plant or mammalian genes encoding polypeptides such as antibodies and antibody fragments. Expression constructs, transformed plants and cells and various methods are provided in accordance with various aspects of the invention.

Plants offer a number of potential advantages for the production of polypeptides of industrial or medical utility, such as mammalian proteins, including antibody molecules, whether complete antibodies or fragments such as single-chain Fv antibody molecules (scFv's), and fusion proteins. Synthesis of functional antibodies in transgenic plants was first demonstrated by Hiatt et al. (Nature (1989) 342: 76-78) and subsequently single chain fragments have been successfully expressed in leaves of tobacco and Arabidopsis plants (Owen et al. (1992) Bio/Technology 10: 790-794; Artsaenko et al. (1995) The Plant J 8: 745-750; Fecker et al. (1996) Plant Mol Biol 32: 979-986). Fiedler et al. (Bio/Technology (1995) 13: 1090-1093) have shown the feasibility of long-term storage of scFv's in tobacco seeds. Almost exclusively, such work has been in dicotyledonous plants. However, monocot crop plants such as wheat and rice have advantages over dicots such as tobacco in not containing noxious chemicals such as alkaloids. This increases possibilities for safe production of polypeptides for pharmaceutical use. Furthermore, crop plants are of particular significance in food contexts, allowing for provision of “functional foods” which may have potential health benefits. An exemplary application is anti-dental caries antibodies, e.g. as expressed by Ma et al. (Eur J Immunol 24: 131-138 (1994); Plant Physiology 109, 341-346 (1995); Science (1995) 268, 716-719) in transgenic tobacco (not a functional food as such).

As far as the present inventors are aware the only experimental example of expression of an antibody or other mammalian protein in a monocot is disclosed in WO98/10062 (Monsanto), published Mar. 12, 1998. This document reports expression of antibody light and heavy chains from separate plasmids in transgenic maize plants, under the control of the rice glutelin-1 promoter.

The present inventors have devised various expression constructs for mammalian genes such as antibodies to be produced in transgenic plants, especially monocots, preferably barley, rice, corn, wheat, oat, sorghum, more preferably wheat, rice. As noted, no-one has previously reported successful expression of such genes in these plants. Experimental evidence described below shows various advantages and benefits from use of different aspects of the expression constructs.

In one aspect of the present invention it has been found that levels of antibody expression in monocots can be enhanced by employing an endoplasmic reticulum (ER) retention signal. Such a signal is a peptide tag usually including the amino acid sequence Lys Asp Glu Leu (KDEL) (SEQ ID NO. 2) or His Asp Glu Leu (HDEL)(SEQ ID NO. 4). Artsaenko et al. employed KDEL in expression of a single-chain Fv antibody against abscisic acid in the dicot tobacco (The Plant J. (1995) 8:745-750), but this has not previously been shown to be functional in monocots.

In another aspect of the present invention, various leader peptide sequences have been found to enhance antibody expression in plants, especially monocots. None of these have previously been shown to be effective in plants. Details are provided below, but no measurable expression of antibody molecule was found in rice calli using a construct without a leader peptide sequence.

In another aspect of this invention suitable membrane anchor peptide sequence, e.g., human T cell receptor transmembrane domain, glyco-phosphatidyl inositol anchors, immunoglobulin superfamily membrane anchors or tetraspan family members, may be included in the polypeptide to allow integration of the polypeptide into cellular membranes.

In a still further aspect of the present invention various 5′ untranslated regions (5′UTR) have been employed in expression of antibody molecules in plants in particular the chalcone synthase and omega 5′UTR's (see below for details). Again, none of these have previously been shown to be effective as demonstrated herein in plants, especially monocots.

Various aspects of the invention provide nucleic acid constructs and vectors including one or more of these elements, transformed host cells, which may be microbial or plant, transgenic callus and suspension cultures and plants and various methods for provision or use of such constructs, vectors, host cells, cultures and plants in production of non-plant, particularly eukaryotic polypeptides, such as antibody molecules.

### BRIEF DESCRIPTION OF THE FIGURE

FIG. 1 shows an schematic of the components in expression constructs according to the present invention. In addition to the promoter and the gene of interest, one or more of the other elements (5′UTR, leader peptide, signal (e.g. KDEL), 3′UTR, pA—polyadenylation signal) may be included and the present invention provides any combination of these elements.

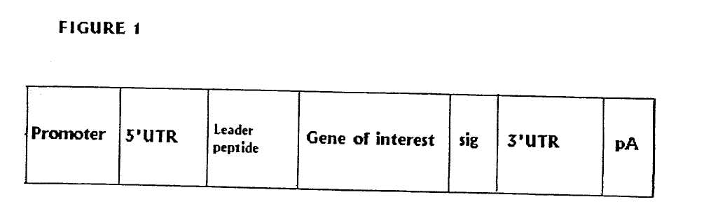

In accordance with a first aspect of the present invention there is provided a plant cell or seed, preferably monocot, containing a polypeptide produced by expression within the cell or seed from an expression cassette including a coding sequence for the polypeptide fused to an endoplasmic reticulum (ER)retention signal.

The retention signal may be a peptide with the amino acid sequence KDEL (SEQ ID NO. 2) or HDEL (SEQ ID NO. 4). KDEL may be encoded by the nucleotide sequence AAA GAT GAG CTC (SEQ ID NO. 1) and HDEL may be encoded by CAT GAT GAG CTC (SEQ ID NO. 3). Other sequences encoding the amino acids but differing from these nucleotide sequences by virtue of degeneracy of the genetic code may be employed. The KDEL or HDEL encoding sequence may be operably linked to a coding sequence for the polypeptide to provide a coding sequence for a fusion of the polypeptide and ER retention signal. Generally the retention signal is placed at the C-terminus of the polypeptide. The ER-retention signal may be preceded by a linker sequence, such as (Gly)₄Ser (SEQ ID NO. 5) and/or Arg Gly Ser Glu (RGSE)(SEQ ID NO. 6) (Wandelt et al. (1992) Plant J. 2(2): 181-192).

In accordance with a second aspect of the present invention there is provided a plant cell or seed, preferably monocot, containing a polypeptide produced by expression within the cell or seed from an expression cassette including a coding sequence for the polypeptide and an 5′ untranslated leader sequence (5′UTR). The 5′UTR may be that of the chalcone synthase gene of petunia (Reimold et al. (1983) EMBO J 2: 1801-1805) or a modified form including one or more additions, deletions, substitutions or insertions of one of more nucleotides, preferably modified to include the T's emboldened in the following sequence:

GAATTCACAACACAAATCAGATTTATAGAGAGATTTATAAAAAAAAAAAAACATATG

(SEQ ID NO. 7). The 5′UTR may be that of the TMV omega gene (Callie et al. (1992) NAR 20: 4631-4638) or a modified form including one or more additions, deletions, substitutions or insertions of one of more nucleotides, preferably including modifications as described by Schmitz et al. (1996) NAR 24: 257-263; incorporated herein by reference. The omega untranslated leader sequence from the U1 strain of TMV is (at the RNA level):

GUAUUUUUACAACAAUUACCAACAACAACAAACAACAAACAACAUUACAAUUACUAUUUAC AAUUACAATG (SEQ ID NO. 8). (Obviously the “U's” are “T's” at the DNA level. The initiation codon is indicated at the end of the sequence here.) One modification preferred in accordance with embodiments of the present invention is to alter the underlined AUU to AGG. Additionally, one or both of the underlined A's may be deleted. A preferred modified sequence is:

GUAUUUUUACAACAAUUACCAACAACAACAACAACAACAACAUUACAAUUACUAUUUACAA GGACCAUGG (SEQ ID NO. 9). In addition to the preferred AUU->AGG modification, this also includes a near-Kozak sequence ACCAUGG, where the AUG is the initiation codon.

In accordance with a third aspect of the present invention there is provided a plant cell or seed, preferably monocot, containing a polypeptide produced by expression within the cell or seed from an expression cassette including a coding sequence for the polypeptide and a leader peptide. A leader peptide may be used to direct the product to a particular cellular compartment. The leader peptide may be of mammalian origin, and may be murine, such as an immunoglobulin light or heavy chain leader peptide. The nucleotide sequence used in the construct to encode the leader peptide may be codon optimised for expression in the plant of interest, preferably monocot, e.g. corn, rice or wheat. A preferred leader peptide useful in accordance with this aspect of the present invention is that of the TMV virion specific mAb24 of Voss et al. (Mol Breed (1995) 1: 39-50) (incorporated herein by reference). Modified forms may be employed. As with other elements for use in expression cassettes in accordance with various aspects of the present invention, the coding sequence may be codon optimised for monocot codon usage according to Angenon et al. (FEBS (1990) 271:144-146) (incorporated herein by reference). The leader peptide may be vacuole targeting signal, such as the leader peptide of a strictosidine synthase gene, e.g. that of the Catharanthus roseus strictosidine synthase (McKnight et al., Nucleic Acids Research (1990), 18, 4939; incorporated herein by reference) or of Rauwolfia serpentina strictisodine synthase (Kutchan et al. (1988) FEBS Lett 237 40-44; incorporated herein by reference). For a review of vacuole targeting sequences see Neuhaus (1996) Plant Physiol Biochem 34(2) 217-221. The leader peptide may be a chloroplast targeting signal such as of the pea rubisco leader peptide sequence (Guerineau et al. (1988) NAR 16 11 380) (incorporated herein by reference). For a review of chloroplast targeting peptides see van Heijne et al. (Eur J Biochem (1989) 180: 535-545) or Kavanagh et al. (MGG (1988) 215: 38-45) or Karlin-Neumann et al. (EMBO J (1986) 5: 9-13) (all incorporated herein by reference). The leader peptide may be a 5′ sequence of a seed storage protein, dicot or monocot, causing transport into protein bodies, such as the Vicia fabia legumin B4 leader (Baeumlein et al. Mol Gen Genet (1991) 225: 121-128) (incorporated herein by reference).

Suitable membrane anchor sequences, enabling the integration of secretory recombinant antibody fusion proteins and parts thereof in the plasma membrane, include the human T cell receptor transmembrane domains (Gross and Eshhar, (1992) “Endowing T Cells with Antibody Specificity Using Chimeric T Cell Receptors,” Faseb J., 6:3370-3378; incorporated herein by reference), glyco-phosphatidyl inositol (GPI) anchors (Gerber et al., (1992) “Phosphatidylinositol Glycan (PI-G) Anchored membrane Proteins. Amino Acid Requirements Adjacent to the Site of Cleavage and PI-G Attachment in the COOH-Terminal Signal Peptide” J. Biol. Chem., 267:12168-12173; incorporated herein by reference), immunoglobulin superfamily membrane anchors, tetraspan family members (Tedder and Engel, (1994) “CD20: A Regulator of Cell-Cycle Progression of B Lymphocytes”, Immunol. Today, 15:450-454); Wright and Tomlinson, (1994) “The Ins and Outs of the Transmembrane 4 Superfamily”, Immunol. Today, 15:588-594; both incorporated herein by reference) and any transmembrane sequence(s) from a known protein or synthesized sequences that have a similar function and can be included in the target protein by recombinant DNA technology. Fusion of a protein to these sequences would permit display of the recombinant protein on the lumenal face of organelles of the secretory or endocytic pathway or the plant cell membrane.

In addition, the antibodies or parts thereof, or the recombinant antibody fusion proteins, or parts thereof, may be targeted to cell membranes where they could face the cytosolic side of the membrane. Suitable targeting sequences for cytoplasmic display, include the transmembrane domains of: KAR1, for nuclear membrane integration (Rose and Fink (1987) “KAR1, a Gene Required for Function of Both Intranuclear and Extranuclear Microtubules in Yeast”, Cell, 48:1047-1060;incorporated herein by reference), middle-T antigen (Kim et al., (1997) “Evidence for Multiple Mechanisms for Membrane Binding and Integration via Carboxyl-Terminal Insertion Sequences”, Biochemistry, 36:8873-8882;incorporated herein by reference), for plasma membrane integration and cytochrome b5, for ER membrane integration (Kim et al., (1997)). C-terminal linkages to fatty acids using consensus amino acid sequences leading to post translational prenylation, farnesylation, palmitoylation, myristoylation or ankyrin sequence motifs can also be used. This cytoplasmic display method has the significant advantage that the recombinant proteins can be localized at the site of intracellular pathogen replication, where they will have the most potent effect. In addition, membrane localization of proteins stabilizes the protein and reduces the effect of C-terminal protein degradation in vivo.

Suitable membrane anchor sequences, enabling the integration of recombinant antibody fusion proteins and parts thereof in the plasma membrane, include the human T cell receptor transmembrane domains (Gross and Eshhar, (1992)), glyco-phosphatidyl inositol (GPI) anchors (Gerber et al., (1992)), immunoglobulin superfamily membrane anchors, tetraspan family members (Tedder and Engel, (1994); Wright and Tomlinson, (1994)) and any transmembrane sequence(s) from a known protein or synthesized sequences that have a similar function and can be included in the target protein by recombinant DNA technology. In addition, the antibodies or parts thereof, or the recombinant antibody fusion proteins, or parts thereof, may be targeted to cell membranes where they could face the cytosoloic side of the membrane. Suitable targeting sequences for cytoplasmic display, include the transmembrane domains of: KAR1, for nuclear membrane integration (Rose and Fink (1987)), middle-T antigen (Kim et al., (1997)), for plasma membrane integration and cytochrome b5, for ER membrane integration (Kim et al., (1997)). C-terminal linkages to fatty acids using consensus amino acid sequences leading to post translational prenylation, farnesylation, palmitoylation, myristoylation or ankyrin sequence motifs can also be used.

Recombinant antibodies can be fused to different transmembrane anchors to improve the expression levels and stability of these molecules inside the plant cell, by targeting the expressed recombinant protein to cell membranes in various orientations. This can be-accomplished by adding: a) C-terminal localization sequences to target and integrate recombinant cytosolic proteins with N-terminal leader peptides into the bilayer of cellular membranes, thus facing to the plant apoplast. Suitable membrane localization sequences include the human T cell receptor chain transmembrane domain and the human platelet derived growth factor receptor (PDGFR) transmembrane domain, glyco-phospatidyl inositol (GPI) anchors, immunoglobulin superfamily membrane anchors and any transmembrane sequence(s) from a known protein or synthesized sequences that have a similar function and can be included in the target protein by recombinant DNA technology. b) Amino terminal transmembrane proteins with either dual or tetrameric plasma membrane spanning domains to expose both the N- and C-termini of secretory recombinant proteins to the cytosol. This can be achieved by using suitable members of the tetraspan family including CD9, CD20, CD81 and the In-Hc-Ic dualspan typeII-IV hybrid of the MHC invariant chain and H-2d hybrid protein. This method enables the orientation of a secreted and membrane anchored antibody construct with its N- and C-terminus into the cytosol. Alternatively, fusions to SNAP-25 can be used for the same orientation. c) C-terminal anchor sequences to target and integrate recombinant cytosolic proteins without N-terminal leader peptides into the bilayer of endomembranes posttranslationally. Suitable targeting sequences include transmembrane domains of KAR1 for nuclear membrane integration (Rose and Fink (1987) “KAR1, a Gene Required for Function of Both Intranuclear and Extranuclear Microtubules in Yeast”, Cell, 48:1047-1060), middle-T antigen for plasma membrane integration (Kim et al., (1997)), and cytochrome b5 for ER membrane integration (Kim et al., (1997)). d) Addition of consensus motifs to the protein that permit C-terminal linkages to fatty acids by prenylation, farnesylation, palmitoylation, myristoylation in the cytosol which then lead to membrane integration. e) Addition of ankyrin sequence motifs (Lambert and Bennett, (1993) “From Anemia to Cerebellar Dysfunction. A Review of the Ankyrin Gene Family”, Eur. J. Biochem., 211:1-6); Peters and Lux, (1992) “Ankyrins: Structure and Function in Normal Cells and Hereditary Spherocytes”, Semin Hematol., 30:85-118; both incorporated herein by reference).

One aspect of this invention is a cereal plant cell or seed containing a mammalian protein produced by expression within the cell or seed from an expression cassette comprising a coding sequence for the protein.

In a further aspect, the present invention provides a corn plant cell or seed containing a mammalian protein produced by expression within the cell or seed from an expression cassette including a coding sequence for the protein.

In a further aspect, the present invention provides a rice plant cell or seed containing a mammalian protein produced by expression within the cell or seed from an expression cassette including a coding sequence for the protein.

A still further aspect of the invention the present invention provides a wheat plant cell or seed containing a mammalian protein produced by expression within the cell or seed from an expression cassette including a coding sequence for the protein.

In further aspects of the present invention there are provided methods for the production of plant cells in accordance with the aspects disclosed above, the methods including introducing into a plant cell nucleic acid including the specified expression construct. Suitable techniques for this, including for vector construction, plant cell transformation, and plant regeneration are discussed below.

Thus, for example, one of these aspects of the invention provides a method including introducing into a plant cell, especially monocot, nucleic acid including an expression cassette including a coding sequence for a polypeptide of interest fused to an endoplasmic reticulum (ER) retention signal. Introduction of nucleic acid into cells may be referred to as “transformation” and resultant cells may be referred to as “transgenic”. This is without limitation to any method or means used to introduce the nucleic acid into the cells.

A transformed cell may be grown or cultured, and further aspects of the present invention provide a suspension culture or callus culture including such cells. As noted below, further aspects provide plants and parts thereof, and methods of production of plants by transformation of cells and regeneration.

It should be noted that plant cells transiently expressing the desired polypeptide following transformation with the appropriate expression cassette are provided by the present invention, but a further aspect provides a method of making a plant cell, preferably monocot, including an expression cassette as disclosed, the method including:

(i) introducing a nucleic acid vector suitable for transformation of a plant cell and including the expression cassette into the plant cell, and,

(ii) causing or allowing recombination between the vector and the plant cell genome to introduce the expression cassette into the genome.

In a still further aspect the present invention provides a method of making a plant, the method including:

(i) making plant cells as disclosed; and

(ii) regenerating a plant from said plant cells or descendants thereof. Such a method may further include cloning or propagating said plant or a descendant thereof containing the relevant expression cassette within its genome.

In various embodiments of the present invention the cell or seed is actively producing the polypeptide or protein.

The expressed polypeptide is preferably a eukaryotic, non-plant protein, especially of mammalian origin, and may be selected from antibody molecules, human serum albumin (Dugaiczyk et al. (1982) PNAS USA 79: 71-75(incorporated herein by reference), erythropoietin, other therapeutic molecules or blood substitutes, proteins within enhanced nutritional value, and may be a modified form of any of these, for instance including one or more insertions, deletions, substitutions and/or additions of one or more amino acids. (The coding sequence is preferably modified to exchange codons that are rare in monocots in accordance with principles for codon usage.)

In preferred embodiments of the present invention, a mammalian protein is an antibody molecule, which includes an polypeptide or polypeptide complex including an immunoglobulin binding domain, whether natural or synthetic. Chimaeric molecules including an immunoglobulin binding domain fused to another polypeptide are therefore included. Example binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341, 544-546 (1989)incorporated herein by reference)) which consists of a VH domain; (v) isolated CDR regions; (vi) F(ab′)2 fragments, a bivalent fragment including two linked Fab fragments (vii) single chain Fv molecules (scFv), wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (viii) bispecific single chain Fv dimers (PCT/US92/09965) and (ix) “diabodies”, multivalent or multispecific fragments constructed by gene fusion (WO94/13804; P. Holliger et al Proc. Natl. Acad. Sci. USA 90 6444-6448, 1993)(all incorporated herein by reference). Monospecific but bivalent diabodies can be produced by expression from a single coding sequence, wherein the polypeptides associate to form dimers including two antigen-binding sites. Bispecific diabodies are formed by association of two different polypeptides, expressed from respective coding sequences.

Where the desired product is a two-chain or multi-chain polypeptide complex (e.g. Fab molecule or bispecific diabody), the expression cassettes may be introduced into plant cells in accordance with the present invention on the same vector or on separate vectors. In one particular aspect of the invention a plant cell, preferably monocot, is transformed separately with four vectors, each including nucleic acid encoding one of the four chains of a secretory antibody, namely the heavy chain, light chain, secretory component and J chain.

The product may be a fusion protein including different proteins or protein domains. For example, certain embodiments of the present invention relate to provision of fusion proteins in which an antibody molecule (such as a scFv molecule or one or both chains of a multimeric antibody molecule such as an Fab fragment or whole antibody) is fused to a non-antibody protein domain, such as interleukin 2, alkaline phosphatase, glucose oxidase (an example of a biological response modifier), green fluorescent protein (an example of a colorimetric label). The non-antibody molecule may be fused to the antibody component at the latter's N- or C-terminus.

Those skilled in the art are well able to construct vectors and design protocols for recombinant gene expression in plants. Suitable vectors can be chosen or constructed, containing appropriate regulatory sequences, including promoter sequences, terminator fragments, polyadenylation sequences, enhancer sequences, marker genes and other sequences as appropriate. For further details see, for example, Molecular Cloning: a Laboratory Manual: 2nd edition, Sambrook et al, 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1992. The disclosures of Sambrook et al. and Ausubel et al. are incorporated herein by reference. Specific procedures and vectors previously used with wide success upon plants are described by Bevan (Nucl. Acids Res. 12, 8711-8721 (1984)) and Guerineau and Mullineaux (1993)(Plant transformation and expression vectors. In: Plant Molecular Biology Labfax (Croy RRD ed) Oxford, BIOS Scientific Publishers, pp 121-148).

Selectable genetic markers may be used consisting of chimaeric genes that confer selectable phenotypes such as resistance to antibiotics such as kanamycin, hygromycin, phosphinotricin, chlorsulfuron, methotrexate, gentamycin, spectinomycin, imidazolinones and glyphosate.

The vector backbone may be pUC (Yanisch-Perron et al. (1985) Gene 33: 103-119) or pSS (Voss et al. (1995) Mol Breed 1: 39-50).

The expression cassette employed in accordance with aspects of the present invention may include the coding sequence under the control of an externally inducible gene promoter to place expression under the control of the user. A suitable inducible promoter is the GST-II-27 gene promoter which has been shown to be induced by certain chemical compounds which can be applied to growing plants. The promoter is functional in both monocotyledons and dicotyledons. The GST-II-27 promoter is also suitable for use in a variety of tissues, including roots, leaves, stems and reproductive tissues.

Other suitable promoters include any constitutive promoter and any seed-specific promoter. Examples include the maize ubiquitin promoter and intron (U.S. Pat. No. 5,510,474), CaMV 35S promoter (Gardner et al. (1981) NAR 9: 2871-2888), and the wheat low molecular weight glutenin promoter (Colot et al. (1987) EMBO J 6: 3559-3564).

A polyadenylation signal such as the NOS terminator may be used (Depicker et al. (1982) J. Mol Appl Genet 1: 499-512). A 3′ UTR such as the modified sequence of TMV as described by Voss et al. (Mol. Breed. (1995) 1:39-50) may be used.

When introducing a chosen gene construct into a cell, certain considerations must be taken into account, well known to those skilled in the art. The nucleic acid to be inserted should be assembled within a construct which contains effective regulatory elements which will drive transcription. There must be available a method of transporting the construct into the cell. Once the construct is within the cell membrane, integration into the endogenous chromosomal material either will or will not occur. Finally, as far as plants are concerned the target cell type may be such that cells can be regenerated into whole plants, although as noted suspension cultures and callus cultures are within the present invention.

A plant cell or seed according to the present invention may be comprised in a plant or part (e.g. leaf, root, stem) or propagule thereof.

Plants which include a plant cell according to the invention are also provided, along with any part or propagule thereof, seed, selfed or hybrid progeny and descendants. A plant according to the present invention may be one which does not breed true in one or more properties. Plant varieties may be excluded, particularly registrable plant varieties according to Plant Breeders' Rights. It is noted that a plant need not be considered a “plant variety” simply because it contains stably within its genome a transgene, introduced into a cell of the plant or an ancestor thereof.

In addition to a plant, the present invention provides any clone of such a plant, seed, selfed or hybrid progeny and descendants, and any part of any of these, such as cuttings, seed. The invention provides any plant propagule, that is any part which may be used in reproduction or propagation, sexual or asexual, including cuttings, seed and so on. Also encompassed by the invention is a plant which is a sexually or asexually propagated off-spring, clone or descendant of such a plant, or any part or propagule of said plant, off-spring, clone or descendant.

Plants transformed with an expression cassette containing the desired coding sequence may be produced by various techniques which are already known for the genetic manipulation of plants. DNA can be transformed into plant cells using any suitable technology, such as a disarmed Ti-plasmid vector carried by Agrobacterium exploiting its natural gene transfer ability (EP-A-270355, EP-A-0116718, NAR 12(22) 8711 - 87215 1984), particle or microprojectile bombardment (U.S. Pat. No. 5100792, EP-A-444882, EP-A-434616) microinjection (WO 92/09696, WO 94/00583, EP 331083, EP 175966, Green et al. (1987) Plant Tissue and Cell Culture, Academic Press), electroporation (EP 290395, WO 8706614 Gelvin Debeyser—see attached) other forms of direct DNA uptake (DE 4005152, WO 9012096, U.S. Pat. No. 4,684,611), liposome mediated DNA uptake (e.g. Freeman et al. Plant Cell Physiol. 29: 1353 (1984)), or the vortexing method (e.g. Kindle, PNAS U.S.A. 87: 1228 (1990d)(all incorporated herein by reference). Physical methods for the transformation of plant cells are reviewed in Oard, 1991, Biotech. Adv. 9: 1-11.

Agrobacterium transformation is widely used by those skilled in the art to transform dicotyledonous species. Recently, there has been substantial progress towards the routine production of stable, fertile transgenic plants in almost all economically relevant monocot plants (Toriyama, et al. (1988) Bio/Technology 6, 1072-1074; Zhang, et al. (1988) Plant Cell Rep. 7, 379-384; Zhang, et al. (1988) Theor Appl Genet 76, 835-840; Shimamoto, et al. (1989) Nature 338, 274-276; Datta, et al. (1990) Bio/Technology 8, 736-740; Christou, et al. (1991) Bio/Technology 9, 957-962; Peng, et al. (1991) International Rice Research Institute, Manila, Philippines 563-574; Cao, et al. (1992) Plant Cell Rep. 11, 585-591; Li, et al. (1993) Plant Cell Rep. 12, 250-255; Rathore, et al. (1993) Plant Molecular Biology 21, 871-884; Fromm, et al. (1990) Bio/Technology 8, 833-839; Gordon-Kamm, et al. (1990) Plant Cell 2, 603-618; D'Halluin, et al. (1992) Plant Cell 4, 1495-1505; Walters, et al. (1992) Plant Molecular Biology 18, 189-200; Koziel, et al. (1993) Biotechnology 11, 194-200; Vasil, I. K. (1994) Plant Molecular Biology 25, 925-937; Weeks, et al. (1993) Plant Physiology 102, 1077-1084; Somers, et al. (1992) Bio/Technology 10, 1589-1594; WO92/14828). In particular, Agrobacterium mediated transformation is now emerging also as an highly efficient alternative transformation method in monocots (Hiei et al. (1994) The Plant Journal 6, 271-282).

The generation of fertile transgenic plants has been achieved in the cereals rice, maize, wheat, oat, and barley (reviewed in Shimamoto, K. (1994) Current Opinion in Biotechnology 5, 158-162.; Vasil, et al. (1992) Bio/Technology 10, 667-674; Vain et al., 1995, Biotechnology Advances 13 (4): 653-671; Vasil, 1996, Nature Biotechnology 14 page 702)(all incorporated herein by reference).

Microprojectile bombardment, electroporation and direct DNA uptake are preferred where Agrobacterium is inefficient or ineffective. Alternatively, a combination of different techniques may be employed to enhance the efficiency of the transformation process, eg bombardment with Agrobacterium coated microparticles (EP-A-486234) or microprojectile bombardment to induce wounding followed by co-cultivation with Agrobacterium (EP-A-486233).

Following transformation, a plant may be regenerated, e.g. from single cells, callus tissue, leaf discs, immature or A mature embryos, as is standard in the art. Almost any plant can be entirely regenerated from cells, tissues and organs of the plant. Available techniques are reviewed in Vasil et al., Cell Culture and Somatic Cell Genetics of Plants, Vol I, II and III, Laboratory Procedures and Their Applications, Academic Press, 1984, and Weissbach and Weissbach, Methods for Plant Molecular Biology, Academic Press, 1989 (both incorporated herein by reference).

The particular choice of a transformation technology will be determined by its efficiency to transform certain plant species as well as the experience and preference of the person practising the invention with a particular methodology of choice. It will be apparent to the skilled person that the particular choice of a transformation system to introduce nucleic acid into plant cells is not essential to or a limitation of the invention, nor is the choice of technique for plant regeneration.

A further aspect of the present invention provides a method of making a plant cell, preferably monocot, as disclosed involving introduction of a suitable vector including the relevant expression cassette into a plant cell and causing or allowing recombination between the vector and the plant cell genome to introduce the sequence of nucleotides into the genome. The invention extends to plant cells containing nucleic acid according to the invention as a result of introduction of the nucleic acid into an ancestor cell.

The term “heterologous” may be used to indicate that the gene/sequence of nucleotides in question have been introduced into said cells of the plant or an ancestor thereof, using genetic engineering, i.e. by human intervention. A transgenic plant cell, i.e. transgenic for the nucleic acid in question, may be provided. The transgene may be on an extra-genomic vector, such as a cow-pea mosaic viral vector, or incorporated, preferably stably, into the genome.

Following transformation of a plant cell, a plant may be regenerated from the cell or descendants thereof.

Further aspects of the present invention provide the use of an expression cassette with features disclosed herein (for example antibody encoding sequence or sequences fused to a mammalian ER retention signal, a peptide leader, and/or a 5′ UTR as disclosed) in production of a transgenic plant cell and in production of a transgenic plant. Such a cell or plant is preferably monocot.

Transgenic plants in accordance with the present invention may be cultivated under conditions in which the desired product is produced in cells and/or seed of the plant. Cells producing the product may be in an edible part of the plant, such as leaves or fruit.

Following cultivation of plants, they, or parts thereof such as their leaves, seed or fruit, may be harvested and processed for isolation and/or purification of the product. Suitable techniques are available to those skilled in the art. The product may be used as desired, for instance in formulation of a composition including at least one additional component.

Seed may be stored, e.g. for at least six months.

Aspects and embodiments of the present invention will now be illustrated by way of experimental exemplification. Further aspects and embodiments of the present invention will be apparent to those skilled in the art.

### EXAMPLE 1

The anti-CEA antibody T84.66 (U.S. Pat. No. 5,081,235) has been used in clinical trials and has a proven potential for therapy and diagnosis.

The present inventors have successfully expressed the T84.66 antigen binding domain in the form of a scFv fragment (scFv84.66) in both rice and wheat. Various untranslated leader and leader peptide sequences were employed. See below for details.

The single-chain fragments were either directed to the apoplast by means of an appropriate mammalian (murine) leader peptide sequence (e.g. construct CH84.66HP (Table 1 construct #1)) or retained in the endoplasmic reticulum by means of an ER retention signal (e.g. construct CH84.66KP(Table 1, construct #5)).

Functional expression of scFv able to bind its antigen was detected by ELISA in rice callus and leaves and in wheat leaves and seeds, both endosperm and embryo.

5/10 wheat plants transformed with CH84.66HP expressed the product in a range of 30-100 ng per gram of leaf material, with an average of 54 ng/g and a maximum of 100 ng/g.

19/30 wheat plants transformed with CH84.66KP expressed the product in a range of 50-700 ng/g, with an average of 243 ng/g and a maximum of 700 ng/g.

14/35 rice calli transformed with CH84.66HP expressed the product in a range of 30-300 ng/g. Four regenerated plants expressed the product in a range of 25-200 ng/g.

7/14 rice calli transformed with CH84.66KP expressed the product in a range of 70-3590 ng/g. Three regenerated plants expressed the product at 1500, 890 and 29000 ng/g leaf material, respectively.

Transformation of rice with construct nr 7, containing the enhanced 35S promoter (2×35S), resulted in seven out of 11 lines expressing scFvT84.66 at levels between 500 and 27000 ng/g leaf tissue. Furthermore, western blot analysis of leaf extracts from selected rice lines transformed with this construct revealed that expressed scFvT84.66 was intact and had the predicted molecular weight.

Table 1 outlines the components of various expression cassettes (see below).

The ubiquitin promoter and the Nos terminator were used in constructs 1 to 6, the enhanced 35S promoter and terminator were used in construct 7.

The results show that use of the ER retention signal enhances accumulation of the protein in wheat and rice plants, that the 5′UTR's are functional in wheat and rice, and that the mammalian leader peptide is functional in wheat and rice.

After six months of storage, the levels of functional, antigen-binding scFv 8466 were not significantly lower than at the time of harvest.

### EXAMPLE 2

The anti-TMV antibody rAb 24 (heavy and light chain EMBL accession numbers X67210 and X67211, respectively) is very well studied. See e.g. Voss et al. (1995) Mol. Breed. 1:39-50 (incorporated herein by reference).

This antibody has been expressed by the inventors in a single-chain Fv format (scFv24) in rice callus and plants. Particularly high amounts of the functional antibody fragment were detected by ELISA (Fischer et al. (1998) Characterization and application of plant-derived recombinant antibodies. In Cunningham C, Porter A (eds), “Methods in Biotechnology, Vol. 3: Recombinant Proteins from Plants: Production and Isolation of Clinically Useful Compounds” Methods in Biotechnology, Vol. 3, 129-142, Humana Press, 1997(incorporated herein by reference)) in callus or rice containing a construct including a C-terminal ER retention signal.

A construct lacking any leader peptide sequence was introduced into rice. No expression was detectable by ELISA in callus tissue or leaves of these transformants.

A construct including the murine leader peptide and encoding scFv24 was used to transform rice and functional scFv was detected by ELISA in callus tissues and leaves. 3/4 rice calli expressed the product.

A further construct including the scFv24 coding sequence and a ER retention signal was expressed in transgenic rice. High levels of functional scFv were detected in callus. 12/25 calli expressed the product in a range of 300-42066 ng/g. One regenerated plant expressed the product at 8635 ng/g.

The results show that the mammalian light chain leader peptide is functional in rice and enhances protein levels as compared to cytosolic expression, and that the ER retention signal is functional in rice and enhances protein levels.

### EXAMPLE 3

The full size chimeric (mouse/human) T84.66 antibody was successfully expressed in rice callus and plants

The genes for heavy and light chain of the antibody were located on two separate plasmids and introduced into plant cells via co-bombardment.

The enhanced 35S promoter was used in all constructs. The heavy and light chain were either both directed to the apoplast by means of an appropriate mammalian (murine) leader peptide sequence (Table 1, constructs 8 and 9) or, alternatively, the heavy chain was retained in the endoplasmic reticulum by means of an ER retention signal (Table 1, construct 10).

Functional expression of T84.66 able to bind its antigen was detected by ELISA in rice callus, leaves and seeds (Table 3). For a positive ELISA reaction, both the light chain and the heavy chain have to be expressed. If light and heavy chains are produced at different levels, the ELISA assay only indicates expression indicative of the lower expression level.

The results show that the genes for the heavy and light chain of a full size antibody can be stably transformed into a plant cell on two separate plasmids. Functional antibody molecules are able to assemble in the plant cell if either both chains are targeted to the apoplast, or if one chain is retained in the ER.

### EXAMPLE 4

The full size anti-TMV antibody rAb 24 was expressed in the apoplast of rice callus cells. The genes encoding the heavy and light chain were both driven by enhanced 35S promoter sequences and present on the same transformation vector. Seven out of 10 rice callus lines expressed functional (antigen binding) full size antibodies at levels between 100 and 50000 ng/g.

The result shows that a functional anti-TMV antibody was produced in rice callus after introducing one plasmid containing the genes encoding heavy and light chain.

### EXAMPLE 5

The anti-TMV antibody rAb 24 was expressed in rice callus and leaves in a Fab (construct 11), F(ab)₂ (construct 12) and bispecific single chain Fv format (construct 13). Various UTR and leader sequences were employed (constructs 11-13; Table 4). The enhanced 35S promoter and 35S terminator were used throughout.

Ten out of 18 rice callus lines transformed with construct 11 expressed the Fab24 fragment, directed to the apoplast, in a range of 30-5200 ng/g. A regenerated transgenic plant expressed the Fab fragment at 2500 ng/g leaf material. Furthermore, western blot analysis of leaf extracts confirmed that expressed Fab 24 was intact and had the predicted molecular weight (double band at 28 kDa).

Three out of 5 rice callus lines containing construct 12 expressed functional (antigen binding) F(ab)₂ antibody fragments directed to the apoplast. The levels of F(ab)₂ measured were in the range of 100-29000 ng/g.

Six out of 8 rice callus lines containing construct nr 13 produced the bispecific single chain fragment of rAb24 in a range of 240 to 31000 ng/g. Two regenerated transgenic plants expressed the biscFv24 fragment in leaves at levels of 2100 or 1200 ng/g, respectively. In this case, an ER retention sequence was attached to the C-terminus of the antibody fragment.

Rice callus lines containing construct 14, encoding the scFv24 fused to the coatprotein of TMV (tobacco mosaic virus), expressed the product at detectable levels. This was determined by ELISAs based on the antigen binding capability of the scFv24.

These results show that various antigen binding fragments, such as Fab fragment, F(ab)₂ fragment, bispecific scFv and scFv fusion proteins can be expressed in callus and leaf tissue of transgenic rice lines.

### EXAMPLE 6

Rice callus tissue was transformed with constructs containing the gene for scFv24 fused to various peptide signals for subcellular targeting. These targeting signals include the N-terminal chloroplast targeting signal of the structural gene for granule-bound starch synthase of potato (van der Leij et al., Mol Gen Gen (1991), 228: 240-248; incorporated herein by reference) and the N-terminal vacuolar targeting signal of strictosidine synthase from Catharanthus roseus (McKnight et al., Nucleic Acids Research (1990), 18, 4939; incorporated herein by reference). In a further construct, a cDNA fragment encoding the constant and transmembrane domain of the human TcRβ chain (Yoshikai et al. Nature (1984), 312: 521-524; incorporated herein by reference) was fused to the coding sequence for scFv24 to obtain membrane anchoring of the product.

Product expression was achieved at levels between 50 and 500 ng/g.

These results show that an antigen binding fragment, such as scFv, can be successful expressed in fusion with signal peptides for targeting to different subcellular compartments.

### EXAMPLE 7

Guy's 13 antibody is a secretory antibody (SigA) with specificity to the streptococcal antigen (SA) I/II cell surface adhesion protein of the oral pathogen Streptococcus mutans (Smith and Lehner (1989) Oral Microbiol Immunol. 4: 153). A secretory form of this antibody has been constructed and used in tobacco (Ma et al. (1995) Science 268: 716; incorporated herein by reference). The molecule consists of IgA dimers associated with the J-chain and the secretory component.

A chimeric mouse/human secretory antibody derived from Guy's 13 was expressed in transgenic rice lines. The four components, namely heavy chain, light chain, J-chain and secretory component, were encoded by four coding sequences, each driven by the maize ubiquitin promoter. The four cassettes were present on four separate plasmids and introduced into the plant cells by co-bombardment.

All coding sequences contained their natural leader peptides for secretion to the apoplast.

Fully assembled SigA was detected in several callus lines, up to a level of 800 ng/g. Fully assembled SigA was also detected in leaf material of a regenerated plant.

The result shows that complex antibodies, such as SigA, can be expressed in callus and leaves of rice following the introduction of the genes encoding the components on separate plasmids.

Materials and Methods

Plasmids and Bacteria

ScFv 84.66 Plasmid Construction

A DNA fragment encoding the single-chain (scFv) protein derived from the anti-CEA antibody T84.66 was amplified by PCR using the construct pUC18-T84.66/212 (Wu et al., 1996 Immunotechnology 2: 21-36; incorporated herein by reference)) as a template, and specific primers introducing NcoI and SalI restriction sites at the 5′ and 3′ ends respectively, for subcloning. The integrity of the scFvT84.66 gene was confirmed by DNA sequencing (ALF, Pharmacia).

The NcoI/SalI amplified T84.66 fragment was subcloned into a pGEM3zf vector containing the 5′ untranslated region of chalcone synthase (CHS 5′ UTR) and the heavy chain leader peptide (muLPH*) from the TMV virion-specific mAb24 (Voss et al., (1995) Mol Breed 1: 39-50). The muLPH* sequence was codon optimised for plant expression according to Angenon et al. (FEBS (1990) 271: 144-146). Also included were either a KDEL motif or a His6 tag 3′ to the T84.66 single-chain fragment as a C-terminal translation modification signal.

The whole cassette, containing either CHS 5′ UTR-muLPH*-T84.66-KDEL or CHS 5′ UTR-muLPH*-T84.66-His6 was recovered with EcoRI and HindIII digestion and subcloned into a pUC19 plasmid containing the maize ubiquitin 1 promoter, intron1 (U.S. Pat. No. 5,510,474; (1990)) and the NOS termination sequence to give the final expression constructs. For co-transformation plasmid pAHC20 was used. This plasmid contains only the bar gene fused to the ubiquitin 1 promoter and intron 1 (Christensen and Quail (1996) Transgen Res 5: 213-218; incorporated herein by reference).

scFv24 Plasmid Constructs for Plant Expression

The heavy and light chain cDNAs of rAb24 (EMBL accession numbers X67210 and X67211, respectively) were used for generation of scFv-cDNAs. The V_{L} and V_{H} fragments were amplified by PCR using domain-specific primers. For each domain one primer contained an overlapping sequence to form the V_{L} and V_{H} connecting linker (marked in italics) by splice overlap extension (SOE) PCR(Horton et al. “Engineering hybrid genes without the use of restriction enzymes; Gene splicing by overlap extension” Gene 77:61-68 (1989); incorporated herein by reference), and was used in conjunction with a primer containing either an EcoRI (V_{L}) or a SalI (V_{H}) restriction site (marked in bold).

The V_{L} domain was amplified using the forward primer P1-front:

5′-GCCGAATTCCATGGACGTCGAGCTGACCCAGTCT-3′ (SEQ ID NO. 10), and the reverse primer P2-back:

5′-CTTTCCGGAACCACTAGTAGAGCCTTTTATCTCCAGCTTGGT-3′ (SEQ ID NO. 11).

The V_{H} domain was amplified using the primers P3-front:

5′-GGTTCCGGAAAGAGCTCTGAAGGTAAAGGTGAGGTCCAGCTGCAGCAG-3′ (SEQ ID NO. 12) and P4-back:

5′-GCCTCTAGACGTCGACTGCAGAGACAGTGACCAG-3′ (SEQ ID NO. 13).

Individual V_{L} and V_{H} fragments were purified and assembled into a scFv fragment by SOE-PCR (Horton et al. (1989)) and subcloned into the EcoRI and SalI restriction sites of a pUC18 derivative, containing a c-myc and His6 sequence. A NdeI restriction site was introduced by PCR using the primer P5L24NL:

5′-GCACACCCGAATTCGGGCCCGGGCATATGCAAATTGTTCTCACCCAGTCT-3′ (SEQ ID NO. 14), to enable cloning of the 5′-untranslated region of chalcone synthase (CHS 5′-UTR) as an EcoRI-NdeI fragment.

The subsequent ligation of the EcoRI-XbaI fragment into the plant expression vector pSS (see below), containing an enhanced 35S promoter and the CaMV termination sequence, resulted in the final construct pscFv24CW, which was used for scFv expression in the cytosol. A second construct (pscFv24CM) was generated by exchanging the 5′ EcoRI-PstI fragment of pscFv24CW with its corresponding region from the full-size light chain cDNA containing the CHS 5′-UTR and the original murine leader peptide sequence of the light chain cDNA of rAb24 to enable scFv secretion into the apoplast.

Plant Material

Plants of Triticum aestivum L., cv Bobwhite, were grown in greenhouse and growthrooms at 15/12° C. day/night temperature and 10 h photoperiod during the first 40 days, followed by maintenance at 21/18° C. day/night temperature and 16 h photoperiod. Plants for insect bioassay were transferred to a heated glasshouse; day length was supplemented with artificial lighting to give a 16 h photoperiod, and temperature was maintained in the range 8-25° C.

Target Tissue and Transformation

Immature embryos were removed and cultured as described (Vasil et al. (1992) Bio/Technology 10: 667-674). After 6 to 7 days, particle bombardment was performed using standard conditions. Thirty to seventy micrograms of coated gold particles/shot were delivered to the target tissue which was incubated on medium containing high osmoticum (0.2 M mannitol and 0.2 M sorbitol) for 5-6 hours prior to and 10-16 hours after bombardment. Plasmids containing the unselected gene and the plasmid containing the bar gene were mixed for co-transformation at a molar ratio of 3:2 and precipitated onto gold particles (Christou et al., 1991 Bio/Technology 9: 957-962; incorporated herein by reference).

Bombarded callus was selected on medium containing phosphinothricin, as described elsewhere (Altpeter et al., 1996, Plant Cell Rep 16: 12-17; incorporated herein by reference).

PAT Assays

PAT activity was assayed using leaf tissue as described before transferring the plants to soil (Vasil et al., (1992) Bio/Technology 10: 667-674).

Production of Monoclonal Antibody and CEA Antigen

The pPIC9K yeast expression vector containing the CEA/NA3 domain and the mAb84.66 was used. The CEA/NA3 protein was expressed in Pichia pastoris strain GS115 (InVitrogen) and purified from the fermentation broth using Ni-NTA affinity chromatography.

The hybridoma cell line T84.66 (Wagener et al., 1983 Journal of Immunology 130: 2308-2315; incorporated herein by reference) was grown in RPMI 1640 (Biochrom) containing 10% fetal calf serum (Biochrom) 25 mM NaHCO₃, 1 mM L-glutamine, 50 μM 2-mercaptoethanol, 24 mM sodium bicarbonate, 50 IU penicillin and 50 μg streptomycin per ml (Gibco) and maintained at 37° C. in a humidified incubator with 7% CO₂. Immunoglobulins from culture supernatants were subjected to affinity chromatography on protein-A HC (BioProcessing). The purity of the mAb preparation was analysed by SDS-PAGE (Laemmli 1970). The presence of CEA-specific antibodies was ascertained by ELISA.

Protein Extraction and ELISA

Extraction of total soluble proteins from leaves and seeds was performed as described by Fischer et al. (1998) (Characterization and application of plant-derived recombinant antibodies. In Cunningham C, Porter A (eds), “Methods in Biotechnology, Vol. 3: Recombinant Proteins from Plants: Production and Isolation of Clinically Useful Compounds”, Methods in Biotechnology, Vol. 3, 129-142, Humana Press Inc., 1997).

Functional T84.66 single-chain antibody was measured in an enzyme linked immunosorbent assay (ELISA) by competition with the full-size murine T84.66 monoclonal antibody. Microtitre plates were coated with 50 ng CEA/NA3 in bicarbonate buffer and blocked with 150 μl bovine serum albumin (1.0% in saline buffer (0.85% NaCl, pH7.2)). Serial dilutions of plant extracts were made using extracts from non-infiltrated control leaves, and 100 μl of each diluted sample, also containing 2.5 ng full-size murine T84.66 antibody was transferred to the CEA/NA3 coated and blocked ELISA plate. Alkaline phosphatase-conjugated Fc specific goat anti-mouse IgG (100 μl of a 1:5000 dilution; Jackson Immunoresearch) was added to each well, and plates were then developed for up to 1 h at 37° C. with 100 μl AP substrate (1 mg ml⁻¹ p-nitrophenlyphosphate, Sigma, in substrate buffer (0.1 M Dietholamine, 1 mM MgCl₂ pH9.8) before reading the absorption at 405 nm using a Spectra Max 340 spectrophotometer (Molecular Devices).

Southern and Northern Blot

DNA was prepared from leaf tissue according to Dellaporta et al., (1984) Maize DNA miniprep. In Malmberg R, Messing J, Sussex I (eds), “Molecular biology of plants. A laboratory course manual”, pp36-37. Cold Spring Harbor Laboratory, Cold Spring Harbor, N.Y.; incorporated herein by reference. 15 μg aliquots of DNA were digested with appropriate restriction endonucleases and subjected to electrophoresis on 0.9% agarose gels. Transfer to nylon membranes and hybridisation were carried out according to standard procedures (Sambrook et al.(1989) Molecular Cloning: A Laboratory Manual. Cold Spring Harbor, N.Y.)

Total RNA was isolated from leaves of transgenic wheat plants, subjected to agarose gel electrophoresis (15 μg per lane) and blotted to a nitrocellulose membrane. 32P-labelled hybridisation probes comprising the coding region of the transgene were prepared using the random primer labelling kit (GIBCO-BRL).

Results

Production and characterisation of transgenic wheat plants Production of transgenic wheat plants by bombardment of immature embryos has been described previously (Altpeter et al., 1997). The gene coding for scFv84.66 and the bar gene, as a selectable marker, were co-transformed into wheat on two separate plasmids. Nine to ten weeks after bombardment, regenerated plantlets were tested for phosphinothricin acetyltransferase (PAT) expression. Forty independent transgenic lines were identified. Thirty lines had been co-bombarded with plasmid pCH84.66KP encoding the scFv antibody with an added KDEL-signal for retention in the ER. The remaining ten lines had been co-bombarded with pCH84.66HP containing the His-tag instead of the KDEL sequence.

Southern blot analysis was carried out on a representative sample of fifteen primary transformants and confirmed the presence of the bar gene in all lines tested. Hybridisation with a probe for the scFv coding sequence revealed the integration of the gene in 11 lines with a co-transformation frequency of ca. 80%. The transgene integration patterns were clearly unique for each line and the complexity of integration varied within the range expected for plants generated via direct gene transfer.

Expression of scFv in Leaves

Extracts of soluble proteins from transgenic leaves were assayed for scFv presence and activity by ELISA. Eighteen out of 27 plants transformed with construct pCH84.66KP showed production levels of up to 700 ng functional active scFv84.66 per g leaf tissue (range: 50-700 ng). The maximum expression level detected in plants containing construct pCH84.66HP was 100 ng per g leaf tissue (range 30-100 ng).

Expression of scFv in Seeds

Mature seeds from the best expressing plants were harvested and extracts of soluble proteins were used for ELISA. Up to 1.5 μg scFv per g seed were determined. These levels of expression exceeded the levels measured in leaves.

Construction of cT84.66 Heavy and Light Chain cDNAs

Splice overlap extension (SOE) PCR was used to obtain full-size size mouse/human chimeric T84.66 light and heavy chain cDNAs, by in frame fusion of the variable VL and VH domains of the mouse mAb T84.66 to the human kappa and IgG1 constant domains of the B72.3 mouse/human chimeric antibody DNAs (Primus et al. (1990) Cancer Immunol Immunother 31, 349-57; incorporated herein by reference). The human constant domains were amplified from plasmids chiB72.3L and chiB72.3H using the following primers: 5′-CTG GAA ATA AAA ACT GTG GCT GCA CCA TCT-3′ (chiB72.3L-I) (SEQ ID NO. 15), 5′-GCC AAG CTT TTT GCA AAG ATT CAC-3′ (chiB72.3L-II) (SEQ ID NO. 16), 5′-ACC GTC TCC TCA GCC TCC ACC AAG GGC CCA-3′ (chiB72.3H-I) (SEQ ID NO. 17), and 5′-GCC AAG CTT GGA TCC TTG GAG GGG CCC AGG-3′ (chiB72.3H) (SEQ ID NO. 18).

The mouse variable domains were amplified from plasmids T84.66L2 (light chain) and T84.66H2 (heavy chain) using the primers: 5′-GGC GAA TTC ATG GAG ACA GAC ACA CTC-3′ (T84.66L-I) (SEQ ID NO. 19), 5′-AGC CAC AGT TTT TAT TTC CAG CTT GGT CCC-3′ (T84.66L) (SEQ ID NO. 20), 5′-GGC GAA TTC ATG AAA TGC AGC TGG GTT-3′ (T84.66H) (SEQ ID NO. 21), 5′-GGT GGA GGC TGA GGA GAC GGT GAC TGA GGT-3′ (T84.66H) (SEQ ID NO. 22).

Chimeric T84.66 light and heavy chain cDNAs obtained by SOE PCR were cloned as EcoRI/HindIII fragments into pUC18, to give the constructs pUC18-“Light” and pUC18-“Heavy”, respectively. All cDNA sequences were confirmed by nucleotide sequencing.

Construction of Full-size cT84.66 Plant Expression Plasmids.

pGEM-3zf was used for cloning the 5′UTR from the omega leader region of tobacco mosaic virus (TMV) (Schmitz et al.(1996) Nucleic Acids Res 24, 257-63), followed by one of the two plant codon optimised leader peptides derived either from the heavy chain (LPH) or from the light chain (LPL) of the murine mAb24 (Voss et al. (1995) Molecular Breeding 1, 39-50), and for cloning the KDEL ER-retention signal sequence, and the 3′UTR from TMV. Chimeric light chain was digested with NcoI/SalI and inserted downstream from the 5′ omega region of TMV and the LPL; chimeric heavy chain was inserted the same way (construct 9), or downstream from the 5′ omega region of TMV and the LPH, and upstream from the KDEL sequence (construct 10). The expression cassettes were cloned between the enhanced 35S promoter and the cauliflower mosaic virus termination region utilising the EcoRI and XbaI restriction sites of the pSS plant expression vector (Voss et al.(1995) Molecular Breeding 1, 39-50).

Construction of Bispecific Single Chain Fv 24 Plant Expression Vectors

To combine scFv24 and the CBHI linker with scFv29 in a bispecific single chain antibody, a cassette arrangement was chosen with restriction sites at the 5′ and 3′ ends of the two scFv and linker sequences.

First, the scFv29 was subcloned into the EcoRI and SalI restriction sites of a pUC18 derivate, containing a His6 sequence (pUC18-scFv29-his). The plasmid pML2 containing the cDNA of the CBHI-linker was used in conjunction with the forward primer CBH-CLA 5′-GCG GAA TTC GTA ATC GAT CCC GGG GGT AAC CGC GGT ACC-3′ (SEQ ID NO. 23) and backward primer CBH-MOD 5′-GCG GAC GTC GCT ATG AGA CTG GGT GGG CCC-3′ (SEQ ID NO. 24) to introduce an EcoRI and ClaI (5′ end) or an AatII (3′ end) restriction site(s) by PCR. The EcoRI and AatII restricted PCR fragment was subcloned into pUC18-scFv29-his (CBHI-scFv29-his). EcoRI and NcoI restriction sites were integrated at the 5′ end of scFv24 (Zimmermann et al. (1998) Molecular Breeding 4, 369-379; incorporated herein by reference) by PCR using the primer SCA25 5′-G CGG AAT TCG GCC ACC ATG GCC CAA ATT GTT CTC ACC CAG TCT-3′ (SEQ ID NO. 25) and a 3′ ClaI site using the primer SCA26 5′-GCG ATC GAT TGC AGA GAC AGT GAC CAG AGT-3′ (SEQ ID NO. 26). Cloning of the EcoRI-ClaI fragment upstream of the CBHI linker in the vector pUC18-scFv29-his gave the biscFv2429 construct pUC18-biscFv2429.

For targeting biscFv2429 to different plant cell compartments, the 5′ EcoRI-StuI fragment of pUC18-biscFv2429, containing the 5′ end of scFv24, was exchanged with its corresponding region from pscFv24CM (Zimmermann et al.(1998) Molecular Breeding 4, 369-379) containing the 5′ untranslated region of the chalcon synthase (CHS 5′-UT) (Voss et al.(1995) Molecular Breeding 1, 39-50) and the original mouse leader sequence of the light chain cDNA. The C-terminal His6 sequence of biscFv2429 was replaced with the ER retention signal KDEL, which was introduced by PCR using the primer KDEL: 5′-ACG CTC TAG AGC TCA TCT TTC TCA GAT CCA CGA GAA CCT CCA CCT CCG TCG ACT GCA GAG ACA GTG ACC AGA GTC CC-3′ (SEQ ID NO. 27) to generate pUC18-biscFv2429-KDEL. The subsequent ligation of the EcoRI-XbaI fragment into the plant expression vector pSS (Voss et al. (1995) Molecular Breeding 1, 39-50), containing an enhanced 35S promoter and the CaMV termination sequence, resulted in the final expression construct biscFv2429-KDEL (Table 4, construct 13), which was used for biscFv2429 expression in the endoplasmic reticulum.

Construction of the Plant Transformation Vector Encoding a scFv24-coatprotein Fusion

The gene fusion partner coat protein (CP) from TMV was amplified by PCR. cDNA was amplified from a cDNA clone from TMV. The forward primers introduced a NcoI restriction site (5′ end) and the backward primers a C-terminal (Gly4Ser)2 linker sequence and an AatII restriction site (3′ end). The following forward and backward primer were used for PCR amplification:

CP-for 5′-ACT GCG CCA TGG CTT ACA GTA TCA CT-3′ (SEQ ID NO. 28), CP-back 5′-CCG TCA GAC GTC AGA ACC TCC ACC TCC ACT TCC GCC GCC TCC AGT TGC AGG ACC AGA GGT CCA AAC CAA ACC-3′ (SEQ ID NO. 29).

The 5′-NcoI and 3′-AatII restricted PCR fragments were subcloned into a pUC18 derivative containing the TMV specific scFv24 (Zimmermann et al. (1998) Molecular Breeding 4: 369-379) flanked by the 5′ untranslated region (omega-sequence) and 3′ untranslated region (Pw sequence) from TMV (Schmitzet al.(1996) Nucleic Acids Res 24: 257-263; Gallie et al., (1994) Gene 142: 159-165).

A C-terminal KDEL-sequence was added to scFv24 by PCR using the backward primer KDEL-back 5′-CCC TCA CTC GAG TTT AGA GCT CAT CTT TCT CAG ATC CAC GAG CGG CCG CAG AAC CTC CAC CTC CGT CGA CTG CAG AGA CAG TGA CCA G-3′ (SEQ ID NO. 30). The subsequent ligation of the EcoRI-AscI fragments into the plant expression vector pSS, containing an double enhanced 35S promoter (Voss et al., 1995), resulted in the final expression construct CP-scFv24K.

Construction of pscFv24-VTS:

The plant codon optimized (for rice, wheat and tobacco) N-terminal vacuolar targeting signal of strictosidine synthase from Catharanthus roseus (McKnight et al., 1990) was added to the scFv24 by PCR using the forward primers VTS5′: 5′-GCC GAA TTC ATA TGG CAA ACT TCT CTG AAT CTA AGT CCA TGA TGG CAG TTT TCT TCA TGT TTT TCC TTC TTC TCC TTT C-3′ (SEQ ID NO. 31) and VTS3′: 5′-ATG TTT TTC CTT CTT CTC CTT TCA TCT AGC TCT TCA AGC TCT TCA TCT TCC ATG GGA CAA ATT GTT CTC ACC CAG TCC C-3′ (SEQ ID NO. 32), which introduce a 5′ EcoRI and NdeI and a NcoI restriction site at the 3′ end of the vacuolar targeting sequence. scFv24CW (Zimmermann et al., 1998) was used as template and a pUC specific oligo as a backward primer. The NdeI and HindIII restricted PCR fragment was subcloned into scFv24CW. The scFv24, cmyc and his6 containing NcoI/HindIII fragment was replaced by an identical but already sequenced fragment. The subsequent ligation of the EcoRI/SalI fragment into the plant expression vector pSS containing a C-terminal c-myc and his6 sequence resulted in the final expression construct pscFv24-VTS.

Construction of pscFv24-CTS:

The plant codon optimized (for rice, wheat and tobacco) N-terminal chloroplast targeting signal of the structural gene for granule-bound starch synthase of potato (van der Leij et al., Mol Gen Gen 1991, 228: 240-248) was added to the scFv24 by PCR using four forward primers: PrimCTS1: 5′-GCC GAA TTC ATA TGG CAT CTA TCA CTG CTT CTC ACC ACT TTG TGT CTA GGT CTC AAA CTT CTC TTG ACA CC-3′ (SEQ ID NO. 33), PrimCTS2: 5′-GGT CTCAAA CTT CTC TTG ACA CCA AAT CTA CCT TGT CTC AGA TCG GAC TCA GGA ACC ATA CTC TTA CTC AC-3′ (SEQ ID NO. 34), PrimCTS3: 5′-TCA GGA ACC ATA CTC TTA CTC ACA ATG GTT TGA GGG CTG TTA ACA AGC TCG ATG GTC TCC AAT CTA GAA C-3′ (SEQ ID NO. 35), PrimCTS4: 5′-CTC GAT GGT CTC CAA TCT AGG ACT AAT ACT AAG GTC ACC CCT AAG ATG GCA TCT AGG ACT GAG ACC AAG AGG C-3′ (SEQ ID NO. 36), and PrimCTS5: 5′-GCA TCT AGG ACT GAG ACC AAG AGG CCA GGA TGC TCT GCT ACC ATT GTT TGC GCC ATG GGA CAA ATT GTT CTC ACC CAG TCT C-3′ (SEQ ID NO. 37), which introduce 5′ EcoRI and 5′ NdeI restriction sites and a NcoI restriction site at the 3′ end of the chloroplast targeting sequence. scFv24CW (Zimmermann et al., 1998) was used as template and a pUC specific oligo as a backward primer. The amplified PCR product was digested with NdeI and HindIII and subcloned into scFv24CW. The scFv24, c-myc and his6 containing NcoI/HindIII fragment was replaced by an identical but already sequenced fragment. The construct was digested with EcoR1 and Sal1 and the EcoRI/SalI fragment containing the scFv sequence was subsequently ligated into the plant expression vector pSS containing a C-terminal c-myc and his6 sequence resulted in the final expression construct pscFv24-CTS.

Construction of scFv24TcRβ:

To generate the fusion construct pscFv24-TcRβ, a cDNA fragment encoding the constant and transmembrane domain of the human TcRβ chain (Yoshikai et al., Nature (1984) 312: 521-524) was PCR amplified from human spleen mRNA (Clontech, Heidelberg, Germany) using the primers 5′-GCC GTC GAC GAG GAC CTG AAC AAG GTG TTC CCA-3′ (SEQ ID NO. 38) and 5′-GCC TCT AGA TCA GAA ATC CTT TCT CTT G-3′ (SEQ ID NO. 39). The primers contained restriction sites SalI and XbaI to enable in frame cloning of the PCR product with scFv24CM. The resulting construct pscFv24-TcRβ was subcloned into the EcoRI and XbaI sites of the plant expression vector pSS (Voss et al., 1995) containing a duplicated CaMV-35S promoter (Kay et al. (1987), Science 236:1299-1302) and the CaMV termination sequence, preceded by a polyadenylation site.

Construction of Plasmids Encoding the SigA Components

A human/mouse hybrid kappa chain was assembled as follows.

An XhoI/HindIII fragment containing the Guy's light variable region, and a HindIII/EcoRI fragment containing the human kappa constant region were ligated together with the native mouse heavy chain leader sequence (muLPH) into a pUC19 plasmid containing the maize ubiquitin 1 promoter, intron 1 and the NOS termination sequence to give the final expression construct.

A KpnI/EcoRI fragment containing the human J chain was ligated into a pUC19 plasmid containing the maize ubiquitin 1 promoter, intron 1 and the NOS termination sequence.

Construction of Fab24 and F(ab)₂ 24

Splice overlap extension (SOE) PCR was used to obtain Fab fragments.

Fusion oligonucleotides 5′-C TGT CCT CCA TGA GCT CAG CAC CCA CAA AAC-3′ (31 mer) (SEQ ID NO. 40) and 5′- GTG CTG AGC TCA TGG AGG ACA GGG GTT GAT-3′ (30 mer) (SEQ ID NO. 41) were used for the SOE of the mouse IgG2b hinge domain and of the 3′-UT of mouse IgG2b in order to obtain Fab-fragments. The final SOE product contains one S-S-bridge (1. cys of the hinge) to the mouse kappa light chain. The second cysteine residue was converted to a TGA stop codon. This oligonucleotide represents the (+)strand and can be used as a backward primer in a PCR to amplify the mouse 3′-UT of IgG2b. The overlap to the mouse hinge domain is 22 bp.

To obtain F(ab)₂ fragments, fusion oligonucleotides 5′-A TGC AAG GAG TGA GCT CAG CAC CCA CAA AGC-3′ (31 mer) (SEQ ID NO. 42)and 5′-TG CTG AGC TCA CTC CTT GCA TGG AGG ACA G-3′ (30 mer) (SEQ ID NO. 43) were used for the SOE of the mouse IgG2b hinge domain and of the 3′-UT of mouse IgG2b in order to obtain F(ab′)₂ fragments. The final SOE product contains two S-S-bridges (1. cys of the hinge to the mouse kappa light chain and the second to the IgG2b heavy chain). The third cys residue was converted to a TGA stop codon. This oligonucleotide represents the (+)strand and can be used as a backward primer in a PCR to amplify the mouse IgG2b in order to obtain mouse F(ab′)₂. The overlap to the mouse hinge domain is 21 bp.

The modified cDNA-Fab and F(ab)₂ fragments were fused to the chalcone synthase (CHS) 5′UTR and subcloned into the plant expression vector pSS, containing the enhanced 35S promoter and CaMV termination signal.

J Chain

A KpnI/EcoRI fragment containing the human J chain was ligated to pMON530. Cloning was confirmed by restriction digest and by PCR analysis.

Secretory Component

Full length native human secretory component was assembled from three sequenced fragments, HuSC2, HuSC3a and the 5′ portion of HuSC (up to the first Acc I site). First, the plasmid containing HuSC was cut with Kpn and religated, to remove the Acc I and EcoR I sites in the vector polylinker. This was confirmed by restriction digest. Plasmids containing HuSC2 and HuSC3 were digested with Xma I and EcoR I, ligated, and selected on chloramphenicol (only one of the two original plasmids was chloramphenicol resistant). Fusion of HuSC2 and HuSC3a was confirmed by restriction digest. An Acc I/EcoR I fragment from the HuSC2/3a clone was used to replace the corresponding fragment in the HuSC clone. The assembled clone was thus made of fully sequenced subfragments, contained Kpn I and Nco I sites at the 5′ end, an EcoR I site at the 3′ end, and no internal Kpn I sites. Correct assembly was confirmed by restriction digests on. The re-assembled Kpn I/EcoR I fragment was ligated to pMON530. Clones were screened by restriction digests. Correct assembly was confirmed by additional restriction digests.

Gamma/Alpha Heavy Chain

A human/mouse hybrid heavy chain was assembled as follows. Plasmids containing the IgG1 C_{H}1-C_{H}2 domains (PHUG) and the Guy's 13 heavy variable region (pGuyHV-2) were both cut with Apa I. A fragment containing the IgG1 C_{H}1-C_{H}2 domains was ligated to the Apa I cut pGuyHV-2. Clones were screened by restriction digest. The resulting hybrid was called pGUY/HUG.

Clones pHuA2 and pHuA3, containing fragments HuA2 and HuA3 respectively, were cut with BspE I and Sac II. The insert fragment released from pHuA3 was ligated to the linearized pHuA2, fusing the C^{H}2-C^{H}3 encoding domains together. Assembly was confirmed by restriction digest. The resulting hybrid was called pHuA2/3.

Plasmid pHuA2/3 was cut with Hind III and Sma I. Plasmid pGUY/HUG was cut with Hind III and Hinc II. The Hua2/3 fragment was ligated to the linearized pGUY/HUG. Correct assembly was confirmed by restriction digests. The resulting clones contain the complete hybrid (glycosylated) heavy chain. The entire cassette was cut out as a Kpn I/Eco RI fragment and cloned into pMON530.

Discussion

The results show that the 5′UTR's, the petunia chalcon synthase and viral omega sequences, are functional in wheat and rice, also the TMV 3′UTR. The mammalian leader peptide sequences, both heavy and light chain, are shown by the results to be functional in cereal callus, leaves and seeds. Use of the ER retention signal produced a higher level of antibody than in the apoplasm. Within the constructs carrying KDEL, CL84.66KP (Construct 4) and OL84.66KP (Construct 6) led to better production levels than analogous constructs containing the murine heavy chain leader peptide, providing indication of advantageous use of leader peptide influencing production level of the expression product in rice.

With the scFv24, expressed in rice callus and plants, pscFv24, lacking any 5′ leader peptide or 3′ signal sequence, did not provide scFv24 at a level detectable using ELISA. A construct containing the gene for scFv including the murine leader peptide (of the light chain) gave detectable levels of scFv24 in transgenic callus lines, although below 200 ng/g. The construct additionally containing a 3′ KDEL sequence yielded the highest levels of scFv, up to 42066 ng/g, range 300-42066 ng/g.

TABLE 1
Constructs containing forms of T84.66.

| Nr | Promoter | cDNA construct | ter |
|---|---|---|---|
| 1 | ubiquitin | 5′UTR(CHS)-muLPH*-scFv84.66-His6-3′UTR(PW-TMV) | NOS |
| | | abbreviation: CH84.66HP | |
| 2 | ubiquitin | 5′UTR(CHS)-muLPL*-scFv84.66-His6-3′UTR(PW-TMV) | NOS |
| | | abbreviation: CL84.66HP | |
| 3 | ubiquitin | 5′UTR(Ome)-muLPH*-scFv84.66-KDEL-3′UTR(PW-TMV) | NOS |
| | | abbreviation: OH84.66KP | |
| 4 | ubiquitin | 5′UTR(CHS)-muLPL*-scFv84.66-KDEL-3′UTR(PW-TMV) | NOS |
| | | abbreviation: CL84.66KP | |
| 5 | ubiquitin | 5′UTR(CHS)-muLPH*-scFv84.66-KDEL-3′UTR(PW-TMV) | NOS |
| | | abbreviation: CH84.66KP | |
| 6 | ubiquitin | 5′UTR(Ome)-muLPL*-scFv84.66-KDEL-3′UTR(PW-TMV) | NOS |
| | | abbreviation: OL84.66KP | |
| 7 | 2x35S | 5′UTR(CHS)-muLPH*-scFv84.66-KDEL-3′UTR(PW-TMV) | 35S |
| 8 | 2x35S | 5′UTR(Ome)-muLPL*-muVL-huCL-3′UTR(PW-TMV) | 35S |
| 9 | 2x35S | 5′UTR(Ome)-muLPH*-muVH-huCH-3′UTR(PW-TMV) | 35S |
| 10 | 2x35S | 5′UTR(Ome)-muLPH*-muVH-huCH-KDEL-3′UTR(PW-TMV) | 35S |

UTR untranslated region
CHS 5′UTR of chalcon synthetase
Ome Omega sequence of TMV (5′-translational enhancer)
muLP murine leader peptide
LPH* heavy chain leader peptide of α-TMV mAB24, codon optimised for tobacco, pea + wheat
LPL* light chain leader peptide of α-TMV mAb24, codon optimised for tobacco, pea + wheat
scFv single chain Fv fragment
84.66 α-CEA antibody T84.66 (binds to A3 domain with high affinity)
His6 histidine 6 for Ni-NTA based affinity chromatography
KDEL C-terminal KDEL motif to enable ER-retention (leads to increased protein accumulation)
stop stop codon
PW pseudoknot region of TMV-wildtype 3′UTR (potential transcriptional and translational enhancer)
TMV tobacco mosaic virus
ubiquitin ubiquitin 1 promoter and intron from maize
2x35S enhanced 35S promoter from cauliflower mosaic virus
NOS terminator from the Nopaline synthase gene of Agrobacterium
35S terminator from cauliflower mosaic virus
muVL murine light chain variable region
muVH murine heavy chain variable region
huCL human light chain constant region
huCH human heavy chain constant regions

TABLE 2
Results of experiments using the cassettes shown in Table 1 to
express the scFv84.66 in rice callus and leaves. The ubiquitin
promoter and the NOS pA were used throughout.

| | callus | leaf | seed |
|---|---|---|---|
| Expression cassette | mean ng/g | mean ng/g | mean ng/g |
| construct 1 | 129 | 59 | 110 |
| construct 2 | n.d. | 61 | n.d. |
| construct 3 | 762 | 1250 | n.d. |
| construct 4 | 1663 | 3030 | 2800 |
| construct 5 | 758 | 10460 | 10050 |
| construct 6 | 1229 | 1460 | n.d. |
| construct 7 | n.d. | 8930 | n.d. |

TABLE 3
Functional expression of T84.66 able to bind its antigen
detected by ELISA in rice callus, leaves and seeds.

| | callus | leaf | seed |
|---|---|---|---|
| Expression cassette | (ng/g) | (ng/g) | (ng/g) |
| Construct 8 + 9 | 100-250 | 250 | 200-300 |
| Construct 8 + 10 | 100-300 | 280 | 200-390 |

TABLE 4
Constructs containing forms of rAb24

| Nr | Promoter | cDNA construct | ter |
|---|---|---|---|
| 11 | 2x35S | 5′UTR(CHS)-muLPL-VL24-CL-3′UTR | 35S |
| | 2x35S | 5′UTR(CHS)-muLPH-VH24-CH1-3′UTR | 35S |
| | | (The two cassettes are in tandem) | |
| 12 | 2x35S | 5′UTR(CHS)-muLPL-VH24-CL-3′UTR | 35S |
| | 2x35s | 5′UTR(CHS)-muLPH-VH24-CH1(2cys)-3′UTR | 35S |
| | | (The two cassettes are in tandem) | |
| 13 | 2x35S | 5′UTR(CHS)-muLPL-VL24-VH24-VL29-VH29-KDEL | 35S |
| 14 | 2x35S | 5′UTR(Ome)-muLPL-CP-scFv24-KDEL-3′UTR(PW-TMV) | 35S |

CP coat protein of TMV

1. A monocotyledonous plant cell or seed containing a mammalian polypeptide produced by expression within the cell or seed from an expression cassette comprising a coding sequence for the polypeptide, in which plant cell or seed there is an additional feature selected from the group consisting of:
(a) the polypeptide is expressed fused to an endoplasmic reticulum (ER) retention signal;
(b) the coding sequence for the polypeptide is preceded in the expression cassette by 5′ untranslated leader sequence (5′UTR);
(c) the polypeptide is expressed fused to a leader peptide;
(d) any combination of two of (a), (b) and (c); and
(e) a combination of (a), (b) and (c).

2. A plant cell or seed according to claim 1 wherein the retention signal is a peptide with the amino acid sequence KDEL (SEQ. ID NO. 2) or HDEL (SEQ. ID NO. 4).

3. A plant cell or seed according to claim 1 wherein the 5′UTR is a chalcone synthase 5′UTR.

4. A plant cell or seed according to claim 3 in which the expression cassette comprises the following 5′UTR sequence:

| | |
|---|---|
| 5′-GAATTCACAACACAAATCAGATTTATAGAGAG | (SEQ.ID NO. 7) |
| ATTTATAAAAAAAAAAAAACATATG-3′. | |

5. A plant cell or seed according to claim 1 wherein the 5′UTR is a TMV omega gene 5′UTR.

6. A plant cell or seed according to claim 5 wherein the 5′UTR has the following RNA sequence:
(SEQ ID NO. 9)
5′-GUAUUUUUACAACAAUUACCAACAACAACAACAACAACAAC
AUUACAAUUACUAUUUACAAGGACCAUGG-3′.

7. A plant cell or seed according to claim 1 wherein the leader peptide is a mammalian leader peptide.

8. A plant cell or seed according to claim 7 wherein the leader peptide is an immunoglobulin light or heavy chain leader peptide.

9. A plant cell or seed according to claim 1 wherein the leader peptide is a vacuole targeting signal

10. A plant cell or seed according to claim 1 wherein the leader peptide is a chloroplast targeting signal

11. A plant cell or seed according to claim 1 wherein the leader peptide causes transport into protein bodies.

12. A plant cell or seed according to claim 1 which is a rice cell or seed.

13. A plant cell or seed according to claim 1 which is a wheat cell or seed.

14. A cereal plant cell or seed containing a mammalian protein produced by expression within the cell or seed from an expression cassette comprising a coding sequence for the protein.

15. A plant cell or seed according to claim 14 that is rice or wheat.

16. A plant cell or seed according to claim 1 wherein an antibody molecule is produced within the cell or seed.

17. A plant cell or seed according to claim 16 wherein the antibody molecule is a single chain Fv antibody fragment.

18. A plant cell or seed according to claim 16 wherein the antibody molecule is a two-chain or multi-chain complex which comprises a plurality of polypeptides and is selected from the group consisting of Fv, Fab, F(ab)₂, diabody, dimeric scFv, whole antibody and four-chain secretory antibody.

19. A plant cell or seed according to claim 18 wherein each polypeptide in said plurality of polypeptides is expressed from a separate expression vector within the cell or seed.

20. A plant cell or seed according to claim 19 wherein the antibody molecule is a four-chain secretory antibody and each of the four chains is expressed from a separate expression vector within the cell or seed.

21. A plant cell or seed according to claim 1 wherein the cell or seed is actively producing the polypeptide.

22. A suspension culture or callus culture comprising a plant cell according to 21.

23. A plant cell or seed according to claim 1 comprised in a plant or plant part.

24. A plant or plant part comprising a plant cell or seed according to claim 1.

25. A method of making a moncotyledonous plant cell comprising an expression cassette as claimed in claim 1, the method comprising:
(i) introducing into a plant cell a nucleic acid suitable for transformation of a plant cell and comprising the expression cassette, and
(ii) causing or allowing recombination between the nucleic acid and the plant cell genome to introduce the expression cassette into the genome.

26. A method according to claim 25 wherein the plant cell is transformed with a plurality of vectors, each of the plurality of vectors comprising an expression cassette comprising a coding sequence for a different polypeptide of a multi-chain complex which comprises a plurality of polypeptides, wherein on production of the polypeptides by expression within the plant cell or descendants thereof the multi-chain complex is formed.

27. A method according to claim 26 wherein the plant cell is transformed with four vectors, each vector encoding a different polypeptide chain of a four-chain secretory antibody, wherein on production of the polypeptides by expression within the plant cell or descendants thereof the secretory antibody is formed.

28. A method according to claim 25 further comprising growing plant cells in plant cell culture to produce the mammalian polypeptide.

29. A method according to claim 28 further comprising isolating and/or purifying the mammalian polypeptide from the plant cell culture.

30. A method of making a plant, the method comprising:
(i) making plant cells according to claim 25, and
(ii) regenerating a plant from said plant cells or descendants thereof.

31. A method according to claim 30 further comprising growing plants to produce the mammalian polypeptide.

32. A method according to claim 31 further comprising isolating and/or purifying the mammalian polypeptide from the plants or parts of the plants.
